(19) United States
(12) Patent Application Publication
(10) Pub. No.: US 2004/0005370 A1
(43) Pub. Date: Jan. 8, 2004

(54) Composition, in particular cosmetic, containing dhea and/or a chemical or biological precursor or derivative thereof , and a metalloproteinase inhibitors

(76) Inventor: Lionel Breton, Versailles (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/332,768

(22) PCT Filed: Jun. 8, 2001
(86) PCT No.: PCT/FR01/01788

(30) Foreign Application Priority Data
Jul. 13, 2000 (FR) 00/09218

(51) Int. Cl. A61K 35/78; A61K 31/704

(52) U.S. Cl. 424/757; 514/26; 514/33

(57) ABSTRACT

The present invention relates to a composition including DHEA and/or a chemical or biological precursor or derivative of the latter, characterized in that it additionally comprises at least one metalloproteinase inhibitor.

The invention also relates to the cosmetic and dermatological uses of this composition, in particular for preventing or treating signs of cutaneous aging.

The present invention relates to a composition including DHEA and/or a chemical or biological precursor or derivative of the latter and at least one metalloproteinase inhibitor, and to the use of said composition, in particular for preventing or treating signs of cutaneous aging.

A thinning of the skin and/or mucous membranes is observed in menopausal women, this thinning being due in particular to changes in the dermis, mainly to a decrease in the level of collagen, which is particularly accentuated under the effect of certain factors, such as ultraviolet rays, the taking of certain medicaments, such as corticoids or vitamin D, and tobacco. The skin then exhibits a soft and wrinkled appearance which conflicts with the current popular preoccupations targeted at retaining for as long as possible, or at recovering, a skin with a youthful appearance.

The importance which there is in combating the decline in collagen in human tissues, in order thus to combat cutaneous aging and its consequences, is thus understood.

To this end, provision has already been made to use DHEA, or dehydroepiandrosterone, to overcome atrophy of the dermis by inhibiting the loss of collagen and of connective tissue (U.S. Pat. No. 5,843,932). In addition, patent U.S. Pat. No. 5,736,537 has disclosed the use by the oral route of DHEA esters, in particular of DHEA salicylate, in regulating atrophy of the skin due to a general deterioration or thinning of the dermis.

Furthermore, the Applicant Company has already demonstrated that DHEA and/or its precursors or derivatives, applied topically, make it possible to prevent or combat other signs of cutaneous aging, namely pigment blemishes (FR 99/12773), a weathered appearance of the skin (FR 00/00349), wrinkles, cutaneous slackening and faded complexion (EP-723 775).

In point of fact, it struck the Applicant Company that the combination of DHEA with a metalloproteinase inhibitor might make it possible to more effectively prevent or treat signs of cutaneous aging and in particular to prevent or treat loss of firmness and/or of suppleness of the skin and/or atrophy of the skin and/or the formation of wrinkles.

The subject matter of the present invention is thus a composition including, in a cosmetically or dermatologically acceptable medium, DHEA and/or a chemical or biological precursor or derivative of the latter, characterized in that it additionally comprises at least one metalloproteinase inhibitor.

DHEA has the following formula (I):

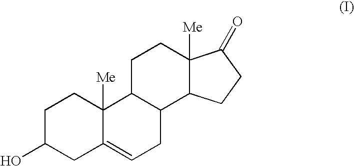

The DHEA which can be used according to the invention is, for example, available from Akzo Nobel.

The term “DHEA precursors” is understood to mean its biological precursors, which are capable of being converted to DHEA during metabolism, and its chemical precursors, which can be converted to DHEA by exogenous chemical reaction. Examples of biological precursors are Δ45-pregnenolone, 17α-hydroxypregnenolone and 17α-hydroxypregnenolone sulfate, without this list being limiting. Examples of chemical precursors are sapogenins or their derivatives, such as diosgenin (or spirost-5-en-3-β-ol), hecogenin, hecogenin acetate, smilagenin and sarsapogenin, and the natural extracts comprising them, in particular fenugreek and extracts of Dioscoreae, such as wild yarn root, without this list being limiting.

The term “DHEA derivatives” is understood to mean both its biological derivatives and its chemical derivatives. Mention may in particular be made, as biological derivatives, of Δ5-androstene-3,17-diol and Δ4-androstene-3,17-dione, without this list being limiting. Mention may in particular be made, as chemical derivatives, of DHEA salts, in particular water-soluble salts, such as DHEA sulfate. Mention may also be made of esters, such as the esters of hydroxycarboxylic acids and of DHEA disclosed in particular in U.S. Pat. No. 5,736,537 or other esters, such as DHEA salicylate, acetate, valerate (or n-heptanoate) and enanthate. Mention may also be made of the DHEA derivatives (DHEA carbamates, DHEA 2-hydroxymalonate esters and DHEA amino acid esters) disclosed in application FR 00/03846 on behalf of the Applicant Company. This list is obviously not limiting.

The DHEA biological and chemical precursors and derivatives will be denoted below by “DHEA analogues”.

The concentration of DHEA and/or analogues in the composition according to the invention is advantageously between 0.0001% and 10% by weight, preferably between 0.001% and 5% by weight, with respect to the total weight of the composition.

The composition according to the present invention includes, in combination with the DHEA or analogue, at least one metalloproteinase inhibitor.

The term “metalloproteinase inhibitor” is understood to mean, according to the invention, any molecule and/or plant or bacterial extract which exhibits an inhibiting activity with respect to at least one of the metalloproteinases expressed and synthesized by and in the skin.

Metalloproteinases are described in particular in the article by Y. Herouy et al., European Journal of Dermatology, No. 3, vol. 10, April-May 2000, pp. 173-180.

The metalloproteinase family is thus composed of several well defined groups based on their similarities in terms of structure and of substrate specificity (see Woessner J. F., Faseb Journal, vol. 5, 1991, 2145). Mention may be made, among these groups, of collagenases intended to decompose fibrillar collagens (MMP-1 or interstitial collagenase, MMP-8 or neutrophil collagenase, MMP-13 or collagenase 3, or MMP-18 or collagenase 4), gelatinases which decompose collagen of type IV or any form of denatured collagen (MMP-2 or gelatinase A (72 kDa), or MMP-9 or gelatinase B (92 kDa)), stromelysins (MMP-3 or stromelysin 1, MMP-10 or stromelysin 2, or MMP-11 or stromelysin 3), the broad spectrum of activity of which applies to proteins of the extracellular matrix, such as glycoproteins (fibronectin, laminin), proteoglycans, and the like, matrilysin (MMP-7), metalloelastase (MMP-12) or membrane metalloproteinases (MMP-14, MMP-15, MMP-16 and MMP-17).

Metalloproteinases (MMPs) are members of a family of proteolytic enzymes (endoproteases) which have a zinc atom coordinated to 3 cysteine residues and a methionine in their active site and which decompose the macromolecular components of the extracellular matrix and of the basal laminae at neutral pH (collagen, elastin, and the like). Very widely distributed in the living world, these enzymes are present, but weakly expressed, in normal physiological situations, such as organ growth and tissue replacement. Their overexpression in man and their activation are related, however, to numerous processes which involve the destruction and the remodeling of the matrix. This results, for example, in an uncontrolled resorption of the extracellular matrix.

Thus, prolonged exposure to ultraviolet radiation, particularly to type A and/or B ultraviolet radiation, has the effect of stimulating the expression of collagenases, particularly of MMP-1. This is one of the components of photoinduced cutaneous aging. In addition, it is known that the activity of MMP-1, MMP-2 and MMP-9 increases with age and that this increase contributes, with a slow down in cell growth, to chronological aging of the skin (WO 98/36742).

Metalloproteinases are produced and secreted in an inactive zymogen form (proenzyme). These zymogen forms are subsequently activated in the extracellular environment by the removal of a propeptide region. The members of this family can activate one another. The activity of MMPs is thus regulated at the level of expression of the genes (transcription and translation), at the level of the activation of the zymogen form or at the level of the local control of the active forms.

The main regulators of the activity of the MMPs are tissue inhibitors of metalloproteinases or TIMPs. However, the expression of MMPs is also modified by growth factors, cytokines, oncogene products (ras, jun) or matrix constituents.

The term “metalloproteinase inhibitor” according to the invention is understood to mean any molecule capable of regulating the activity of MMPs, either at the level of the expression of the genes (transcription and translation), or at the level of the activation of the zymogen form of MMPs, or at the level of the local control of the active forms.

Thus, the metalloproteinase inhibitor according to the invention can be a tissue inhibitor of metalloproteinases (TIMP), such as the peptides known in the prior art under the names TIMP-1, TIMP-2, TIMP-3 and TIMP-4 (Woessner J. F., Faseb Journal, 1991).

In an alternative form, the metalloproteinase inhibitors suitable for use in the present invention can be MMP-1 inhibitors of natural or synthetic origin. The term “natural origin” is understood to mean the metalloproteinase inhibitor, in the pure state or in solution at different concentrations, obtained by various extraction processes from a component, generally a plant, of natural origin. The term “synthetic origin” is understood to mean the metalloproteinase inhibitor, in the pure state or in solution at different concentrations, obtained by chemical synthesis.

According to a particularly preferred embodiment of the invention, use is made of a metalloproteinase inhibitor of natural origin, such as lycopene. This metalloproteinase inhibitor was disclosed in patent application FR 99/12507.

The metalloproteinase inhibitor preferably represents from 10⁻¹² to 5% and preferably from 10⁻¹⁰ to 2% of the total weight of the composition. Of course, if the metalloproteinase inhibitor is present in the form of a solution comprising a plant extract, the person skilled in the art will know to adjust the amount of this solution in the composition according to the invention so as to obtain the above concentration ranges of metalloproteinase inhibitor.

The composition according to the invention can be provided in any pharmaceutical dosage form normally used for topical application to the skin or hair, in particular in the form of an aqueous or oily solution, of an oil-in-water or water-in-oil or multiple emulsion, of a silicone emulsion, of a microemulsion or nanoemulsion, of an aqueous or oily gel, of a liquid, pasty or solid anhydrous product, or of a dispersion of oil in an aqueous phase in the presence of spherules, it being possible for these spherules to be polymeric nanoparticles, such as nanospheres and nanocapsules, or better still lipid vesicles of ionic and/or nonionic type.

This composition can be more or less fluid and have the appearance of a white or colored cream, of an ointment, of a milk, of a lotion, of a serum, of a paste, of a foam or of a gel. It can optionally be applied to the skin in the form of an aerosol. It can also be provided in the solid form, for example in the form of a stick. It can be used as a product for caring for and/or as a product for making up the skin.

In addition, insofar as metalloproteinase inhibitors are already known to induce and/or stimulate hair growth, including that of body hair, and/or to slow down its loss (WO 99/58101), the Applicant Company believes, without wishing to be committed to this theory, that they would be capable of reinforcing the beneficial effect of hair compositions based on DHEA or analogues, in which DHEA is itself known to be effective with respect to canities (FR 99/12773).

Consequently, the composition according to the invention can, in an alternative form, be a hair composition provided in particular in the form of a shampoo or conditioner, for example.

In a known way, the composition of the invention can also comprise the adjuvants usual in the cosmetic and dermatological fields, such as hydrophilic or lipophilic gelling agents, hydrophilic or lipophilic active principles, preservatives, antioxidants, solvents, fragrances, fillers, screening agents, pigments, odor absorbers and coloring materials. The amounts of these various adjuvants are those conventionally used in the fields under consideration and represent, for example, from 0.01 to 20% of the total weight of the composition. These adjuvants, depending upon their nature, can be introduced into the fatty phase, into the aqueous phase, into the lipid vesicles and/or into the nanoparticles. These adjuvants and their concentrations must be such that they do not harm the advantageous properties of the combination of active principles according to the invention.

When the composition according to the invention is an emulsion, the proportion of the fatty phase can range from 5 to 80% by weight and preferably from 5 to 50% by weight with respect to the total weight of the composition. The fatty substances, the emulsifiers and the coemulsifiers used in the composition in the form of an emulsion are chosen from those conventionally used in the field under consideration. The emulsifier and the coemulsifier are preferably present in the composition in a proportion ranging from 0.3 to 30% by weight and preferably from 0.5 to 20% by weight with respect to the total weight of the composition.

Use may be made, as fatty substances which can be used in the invention, of oils and in particular mineral oils (liquid petrolatum), oils of vegetable origin (avocado oil, soybean oil), oils of animal origin (lanolin), synthetic oils (perhydrosqualene), silicone oils (cyclomethicone) and fluorinated oils (perfluoropolyethers). Use may also be made, as fatty substances, of fatty alcohols, such as cetyl alcohol, fatty acids, waxes and gums and in particular silicone gums.

Mention may be made, as emulsifiers and coemulsifiers which can be used in the invention, of, for example, esters of fatty acid and of polyethylene glycol, such as PEG-100 stearate, PEG-50 stearate and PEG-40 stearate; esters of fatty acid and of polyol, such as glyceryl stearate, sorbitan tristearate and the oxyethylenated sorbitan stearates available under the tradenames Tween® 20 or Tween® 60, for example; and their mixtures.

Mention may in particular be made, as hydrophilic gelling agents, of carboxyvinyl polymers (carbomer), acrylic copolymers, such as acrylate/alkyl acrylate copolymers, polyacrylamides, polysaccharides, natural gums and clays and mention may be made, as lipophilic gelling agents, of modified clays, such as bentones, metal salts of fatty acids and hydrophobic silica.

Use may in particular be made, as active principles, of depigmenting agents and keratolytic and/or desquamating agents.

In the event of incompatibility, the active principles indicated above and/or the DHEA or analogue and/or the metalloproteinase inhibitor according to the invention can be incorporated in spherules, in particular ionic or nonionic vesicles and/or nanoparticles (nanocapsules and/or nanospheres), so as to isolate them from one another in the composition.

According to an alternative form of the invention, the composition can be adapted to oral administration. In this case, it can be provided in the form of syrups, of suspensions, of solutions, of emulsions, of capsules, of granules or of tablets, for example.

The daily doses of DHEA or analogues administered by the oral route can be between 1 and 100 mg/day, preferably between 25 and 75 mg/day. The DHEA or analogue is preferably present in the composition according to the invention in an amount which allows it to be administered at a dose of between 50 and 100 mg/day, the said dosage being achieved with the composition taken one or more times, with a preferential unit dose of 50 mg.

In all cases, the composition according to the invention comprises an effective amount of DHEA or analogue and of metalloproteinase inhibitor, sufficient to produce the desired effect, and a physiologically acceptable medium.

The composition according to the invention finds an application in particular in the prevention and treatment of signs of cutaneous aging.

The present invention thus also relates to the cosmetic use of the composition according to the invention for preventing or treating signs of cutaneous aging, in particular for preventing or treating loss of firmness and/or of suppleness of the skin and/or atrophy of the skin and/or the formation of wrinkles.

It also relates to the cosmetic use of this composition in the treatment of the scalp. Finally, it relates to the use of the abovementioned composition in manufacturing a preparation intended for the treatment of the scalp.

The invention will now be illustrated by the following nonlimiting example. In these examples, the amounts are shown as percentage by weight.

### EXAMPLE

### Composition for Topical Application

A care cream (oil-in-water emulsion) is prepared in a way conventional to a person skilled in the art, this cream having the following composition:

| | |
|---|---|
| 6% Lycopene | 10⁻⁴% |
| DHEA | 0.1% |
| Glycerol stearate | 2% |
| Polysorbate 60 (Tween 60 ®, sold by ICI) | 1% |
| Stearic acid | 1.4% |
| Triethanolamine | 0.7% |
| Carbomer | 0.4% |
| Liquid fraction of karite butter | 12% |
| Perhydrosqualene | 12% |
| Fragrance | 0.5% |
| Preservative | q.s. |
| Water | q.s. for 100% |

1. A composition including, in a cosmetically or dermatologically acceptable medium, a sapogenin or a natural extract comprising it and at least one metalloproteinase inhibitor.

2. The composition as claimed in claim 1, characterized in that said sapogenin is chosen from diosgenin, hecogenin, smilagenin and sarsapogenin.

3. The composition as claimed in claim 1, characterized in that said natural extract is chosen from fenugreek and extracts of Dioscoreae.

4. The composition as claimed in claim 3, characterized in that said extract of Dioscorea is a wild yarn root extract.

5. The composition as claimed in any one of the preceding claims, characterized in that it includes from 0.001 to 5% by weight of sapogenin with respect to the total weight of the composition.

6. The composition as claimed in any one of the preceding claims, characterized in that said metalloproteinase inhibitor is a tissue inhibitor of metalloproteinases.

7. The composition as claimed in any one of claims 1 to 6, characterized in that said metalloproteinase inhibitor is an MMP-1 inhibitor of natural origin.

8. The composition as claimed in claim 7, characterized in that said metalloproteinase inhibitor is lycopene.

9. The composition as claimed in any one of the preceding claims, characterized in that it comprises from 10⁻¹⁰ to 2% by weight of metalloproteinase inhibitor with respect to the total weight of the composition.

10. Cosmetic use of the composition as claimed in any one of the preceding claims, for preventing or treating signs of cutaneous aging.

11. Cosmetic use of the composition as claimed in any one of claims 1 to 9 for preventing or treating loss of firmness and/or of suppleness of the skin and/or atrophy of the skin and/or the formation of wrinkles.

12. Cosmetic use of the composition as claimed in any one of claims 1 to 9 in the treatment of the scalp.

13. Use of the composition as claimed in any one of claims 1 to 9 in manufacturing a preparation intended for the treatment of the scalp.
